(19) United States
(12) Patent Application Publication
(10) Pub. No.: US 2005/0159759 A1
(43) Pub. Date: Jul. 21, 2005

(54) Systems and methods for performing minimally invasive incisions

(76) Inventors: Mark Harbaugh, Germantown, TN (US); Albert Pothier, Memphis, TN (US)

Correspondence Address: CHIEF PATENT COUNSEL;SMITH & NEPHEW, INC., 1450 BROOKS ROAD, MEMPHIS, TN, 38116, US

(21) Appl. No.: 10/760,999

(22) Filed: Jan. 20, 2004

(51) Int. Cl. A61B 19/00; A61B 17/32

(52) U.S. Cl. 606/130; 606/167; 128/898

(57) ABSTRACT

Systems and methods for performing minimally invasive incisions may provide guidance for performing a surgical incision by tracking and comparing the position, in real time, of an incision device with a suggested incision path and length. A computer aided surgical navigation system may track the incision device and calculate the suggested incision path and length by tracking the positions and/or orientations of surgical references exhibiting fiducial functionality associated with the incision device and the individual to be incised.

### BACKGROUND

Recovering from a surgical procedure, particularly invasive procedures involving incisions, can be a long and painful experience that may include permanent scarring. Consequently, it may be desirable to minimize the length and size of the incision or incisions made during the procedure. Benefits may include decreased tissue and muscle trauma; less blood loss; quicker recovery time; reduced risk of infection or contamination; less postoperative pain; less scaring and faster and easier rehabilitation.

Typically, the surgeon uses anatomic landmarks, past experience, templates and “rules of thumb” for planning and performing the necessary incision. However, because patients' size and anatomical structure may vary, the ideal incision path and length will also vary from patient to patient. Consequently, traditional methods will not always result in ideal incisions in all situations, which may require the surgeon to revise the incision or to perform a new incision, increasing the invasiveness of the incision.

Computer assisted surgical navigation systems may help surgeons or other users to perform a minimally invasive incision. Several manufacturers currently produce image-guided surgical navigation systems that are used to assist in performing surgical procedures with greater precision. The TREON™ and iON™ systems with FLUORONAV™ software manufactured by Medtronic Surgical Navigation Technologies, Inc. are examples of such systems. The BrainLAB VECTORVISION™ system is another example of such a surgical navigation system. Systems and processes for accomplishing image-guided surgery are also disclosed in U.S. Ser. No. 10/084,012, filed Feb. 27, 2002 and entitled “Total Knee Arthroplasty Systems and Processes”; U.S. Ser. No. 10/084,278, filed Feb. 27, 2002 and entitled “Surgical Navigation Systems and Processes for Unicompartmental Knee Arthroplasty”; U.S. Ser. No. 10/084,291, filed Feb. 27, 2002 and entitled “Surgical Navigation Systems and Processes for High Tibial Osteotomy”; International Application No. US02/05955, filed Feb. 27, 2002 and entitled “Total Knee Arthroplasty Systems and Processes”; International Application No. US02/05956, filed Feb. 27, 2002 and entitled “Surgical Navigation Systems and Processes for Unicompartmental Knee Arthroplasty”; International Application No. US02/05783 entitled “Surgical Navigation Systems and Processes for High Tibial Osteotomy”; U.S. Ser. No. 10/364,859, filed Feb. 11, 2003 and entitled “Image Guided Fracture Reduction,” which claims priority to U.S. Ser. No. 60/355,886, filed Feb. 11, 2002 and entitled “Image Guided Fracture Reduction”; U.S. Ser. No. 60/271,818, filed Feb. 27, 2001 and entitled “Image Guided System for Arthroplasty”; U.S. Ser. No. 10/229,372, filed Aug. 27, 2002 and entitled “Image Computer Assisted Knee Arthroplasty”; and U.S. Ser. No. 10/689,103, filed Oct. 20, 2003 and entitled “Reference Frame Attachment, the entire contents of each of which are incorporated herein by reference as are all documents incorporated by reference therein.

These systems and processes use position and orientation tracking sensors such as infrared sensors acting in a stereoscopic manner or other sensors acting in conjunction with reference structures, including reference transmitters, to track positions of body parts, surgery-related items such as implements, instruments, trial prosthetics, prosthetic components, and virtual constructs or references such as rotational axes which have been calculated and stored based on designation of bone landmarks. Processing capability such as any desired form of computer functionality, whether standalone, networked, or otherwise, takes into account the position and orientation information as to various items in the position sensing field (which may correspond generally or specifically to all or portions or more than all of the surgical field) based on sensed position and orientation of their associated reference structures such as fiducials, reference transmitters, or based on stored position and/or orientation information. The processing functionality correlates this position and orientation information for each object with stored information, such as a computerized fluoroscopic imaged file, a wire frame data file for rendering a representation of an instrument component, trial prosthesis or actual prosthesis, or a computer generated file relating to a rotational axis or other virtual construct or reference. The processing functionality then displays position and orientation of these objects on a screen or monitor. Thus, these systems and processes, by sensing the position of reference structures or transmitters, can display or otherwise output useful data relating to predicted or actual position and orientation of body parts, surgically related items, instruments, implants, and virtual constructs for use in navigation, assessment, and otherwise performing surgery or other operations.

Some of these reference structures or reference transmitters may emit or reflect infrared light that is then detected by an infrared camera. The references may be sensed actively or passively by infrared, visual, sound, magnetic, electromagnetic, x-ray, or any other desired technique. An active reference emits energy, and a passive reference merely reflects energy. In some embodiments, the reference structures have at least three, but sometimes four, markers or fiducials that are tracked by an infrared sensor to determine the position and orientation of the reference and thus the position and orientation of the associated instrument, implant component or other object to which the reference is attached.

The Medtronic imaging systems allow reference structures to be detected at the same time the fluoroscopy imaging is occurring. This allows the position and orientation of the reference structures to be coordinated with the fluoroscope imaging. Then, after processing position and orientation data, the reference structures may be used to track the position and orientation of anatomical features that were recorded with a fluoroscope. Computer-generated images of instruments, components, or other structures that are fitted with reference structures may be superimposed on the fluoroscopic images. The instruments, trial, implant or other structure or geometry can be displayed as 3-D models, outline models, or bone-implant interface surfaces.

U.S. Pat. No. 5,782,842 and International Application No. PCT/GB01/02523 both disclose methods and systems for guiding surgical procedures using computer assisted surgical navigation systems.

International Application No. PCT/GB01/02523, entitled “Method and Apparatus for Guiding a Surgical Instrument,” filed Jun. 8, 2001, discloses a method and apparatus for guiding the tip of a surgical instrument into the body of a mammal along a previously defined path using a computer assisted surgical navigation system. As disclosed in the application, the incision path is determined using a computer assisted surgical navigation system, which includes a reference structure secured to a portion of the individual's anatomy. The system, using information on the position and orientation of the surgical reference, in conjunction with images, such as X-ray images, may calculate an appropriate incision path for reaching a desired target within the individual, such as a brain tumor. Two emitters then project light beams at a target for the path, indicating where the surgeon should direct the instrument. Once the surgeon reaches the target, the light beams can be re-adjusted to the next target along the path.

U.S.P.N. 5,782,842, entitled “Medical Instrument Guidance Apparatus and Method” and issued Jul. 21, 1998 to Kloess et al., discloses guiding a biopsy needle with two fanned beams of light. The intersection of the fanned light beams defines an insertion angle for a biopsy needle. When the biopsy needle is lined up along the proper angle, the needle shines with a certain intensity of light that it does not shine with if the needle is not lined up along the proper angle.

The systems disclosed in U.S.P.N. 5,782,842 and PCT/GB01/02523 may be undesirable. These systems do not use the computer assisted surgical navigation system to track the progress of the surgical instrument during the surgical procedure. Consequently, if the light emitters are inadvertently misaligned, the surgeon may receive inaccurate guidance such that the incision is not performed along the desired incision path and/or length. This potential for imprecision may lead to unnecessarily invasive surgical procedures, which as discussed above, are undesirable. Also problematically, the light emitters of these previous systems may be difficult and time consuming to set-up, properly align and otherwise prepare for the procedure. Additionally, if the light emitters become misaligned, through inadvertent contact or otherwise, it may be necessary to recalibrate the entire system, increasing the time, expense and frustration associated with the procedure.

### SUMMARY

Various aspects and embodiments of the present invention provide systems and methods for performing a minimally invasive skin surface incision using a computer aided surgical navigation system that not only suggests an incision path and length, but also tracks the progress of the actual incision. In some embodiments, the computer aided surgical navigation system also provides feedback to the user if the incision device deviates from the suggested incision path.

The computer aided surgical navigation system may include a sensor adapted to sense the position and/or orientation of surgical references associated with structures to be referenced, such as portions of an individual's anatomy, surgical tools, incision devices, other surgical items, or implants such as orthopaedic implants. The navigation system may also include computer functionality. The computer functionality may be adapted to receive information from the sensors regarding the position and/or orientation of the surgical references and generate information corresponding to the position and/or orientation of the structure associated with the surgical reference.

In some embodiments, the surgical reference is associated with the structure by first securing the surgical reference, either directly or indirectly, to the structure. In some embodiments, the surgical reference is secured directly to the anatomy, preferably the bony anatomy, of an individual. Next, in embodiments where the surgical reference is secured to the bony anatomy of an individual, it is possible that fluoroscopy or other appropriate images as discussed above are obtained. For structures where the computer functionality already has data, such as wire frame data or other data, regarding the structure to be referenced (such as a surgical tool, item or implant), it may not be necessary to image the structure to which the surgical reference is secured.

After the surgical reference is associated with the structure, it may be necessary to register the position and/or orientation of the structure associated with the surgical reference with the computer functionality such that the computer aided surgical navigation system can track the position and/or orientation of the structure. In some embodiments, the structure may be registered with the navigation system simultaneously as the fluoroscope or other images are being obtained. In other embodiments, the structure may be registered with the navigation system simply by using a probe associated with another surgical reference. In still other embodiments, other appropriate techniques may be used to register the structure with the navigation system.

In some embodiments of the present invention, surgical references are associated and registered with both a portion of an individual's anatomy and an incision device. Preferably, at least one surgical reference is associated with the bony anatomy as well as the skin proximate the bony anatomy of an individual. Securing the surgical reference directly to the bony anatomy may allow the surgical reference to also be associated with the skin proximate the bony anatomy to which the surgical reference is secured.

In some embodiments, the incision device may be either a cutting device or a marking device. Surgical references may be associated with these structures in any suitable and/or desirable manner or manners.

In some embodiments, using the information obtained from the sensors sensing the position and orientation of the surgical reference associated with the individual's anatomy and the information generated by the computer functionality concerning the position and orientation of the individual's anatomy, a suggested incision path and length may be calculated, either manually or automatically. In some embodiments, the surgeon may manually specify the desired location, orientation, length and/or path of the incision. In other embodiments, the computer functionality or other functionality associated with the computer aided surgical navigation system may automatically calculate a suggested incision path using one or more appropriate software programs.

In some embodiments of the present invention, before, during or after the actual performance of the incision, the computer aided surgical navigation system may compare the suggested incision path with the actual incision path followed by the incision device. The navigation system may do this by tracking the position and/or orientation of the reference structure associated with the incision device, consequently tracking the position and/or orientation of the incision device itself. In some embodiments, the computer aided surgical navigation system may display the position and/or orientation of the incision device and/or the suggested incision on a display. In other embodiments, the suggested incision path may be projected onto the individual's anatomy using a scanning laser or any other appropriate device.

In some embodiments, in addition to simply comparing the suggested incision path with the position and/or orientation of the incision device, the system may also provide feedback if the incision device deviates from the suggested incision path. Feedback functionality may provide feedback in the form of visual, audible or other appropriate feedback. In some embodiments, the feedback is provided on a display that is displaying the suggested incision path and the actual incision path. The display may be a monitor, a semi-transparent display or any other appropriate display. In other embodiments, the feedback is provided by an image projected onto the skin surface of the individual.

Using systems and methods according to various aspects and embodiments of the present invention may allow surgeons and other users to perform minimally invasive incisions on the skin surfaces of individuals.

### STATEMENT OF THE INVENTION

In accordance with aspects of the present invention, there is provided:

A method of performing an incision on a skin surface using a computer aided surgical navigation system, the computer aided surgical navigation system characterized in that the system includes: a sensor adapted to sense the position and orientation of at least one surgical reference associated with a structure to be referenced; and computer functionality adapted to receive information from the sensor about position and orientation of the surgical reference and generate information corresponding to the position and orientation of the structure to be referenced to which the surgical reference is associated, the method of guiding the surgical incision characterized in that the method includes the steps of: associating at least one first surgical reference with a portion of an individual's bony anatomy and skin proximate the bony anatomy; registering the position and orientation of the portion of the individual's bony anatomy and skin proximate the bony anatomy with the computer aided surgical navigation system such that the computer functionality can generate information corresponding to the position and orientation of the individual's bony anatomy and skin proximate the bony anatomy by receiving information from the sensor sensing the position and orientation of the first surgical reference; calculating a suggested incision path and length based upon the information generated by the computer functionality corresponding to the position and orientation of the individual's bony anatomy and skin proximate the bony anatomy; associating at least one second surgical reference with an incision device, wherein the incision device is a cutting device or a marking device; registering the position of the incision device with the computer aided surgical navigation system such that the computer functionality can generate information corresponding to the position of the incision device by receiving information from the sensor sensing the position and orientation of the second surgical reference; and using the incision device in the performance of the incision, wherein the computer aided surgical navigation system provides guidance based on comparing the suggested incision path and length with the information corresponding to the position and orientation of the incision device as generated by the computer functionality by receiving information from the sensor sensing the position and orientation of the second surgical reference.

A method of performing an incision on a skin surface using a computer aided surgical navigation system further characterized in that the computer aided surgical navigation system providing guidance further comprises a display associated with the computer aided surgical navigation system displaying the suggested incision path and length and the position of the incision device.

A method of performing an incision on a skin surface using a computer aided surgical navigation system further characterized in that displaying the suggested incision path and length and the position of the incision device further comprises a semi-transparent display displaying the suggested incision path and length and the position of the of the incision device or a monitor displaying the suggested incision path and length and the position of the of the incision device.

A method of performing an incision on a skin surface using a computer aided surgical navigation system further characterized in that the method also includes providing feedback if the incision device deviates from the suggested incision.

A method of performing an incision on a skin surface using a computer aided surgical navigation system further characterized in that providing feedback comprises providing a visible warning if the incision deviates from the suggested incision.

A method of performing an incision on a skin surface using a computer aided surgical navigation system further characterized in that providing a visible warning if the incision deviates from the suggested incision comprises displaying a comparison of the suggested incision path and length with at least a portion of a path traveled by the incision device.

A method of performing an incision on a skin surface using a computer aided surgical navigation system further characterized in that the method also includes projecting an image of the suggested incision onto the individual; and wherein the method is further characterized in that providing a visible warning if the incision deviates from the suggested incision comprises altering the image if the incision deviates from the suggested incision.

A method of performing an incision on a skin surface using a computer aided surgical navigation system further characterized in that providing feedback further comprises providing an audible warning if the incision deviates from the suggested incision path.

A method of performing an incision on a skin surface using a computer aided surgical navigation system further characterized in that the method also includes using the incision to access the interior of the individual to install an orthopaedic implant; and installing the orthopaedic implant.

A system for guiding an incision on a skin surface, characterized in that the system includes: a computer aided surgical navigation system, comprising: a sensor adapted to sense the position and orientation of at least one surgical reference associated with a structure to be referenced; and computer functionality adapted to receive information from the sensor about position and orientation of the surgical reference and generate information corresponding to the position and orientation of the structure to be referenced to which the surgical reference is associated; at least one first surgical reference adapted to be associated with a portion of an individual's bony anatomy and skin proximate the bony anatomy such that the position and orientation of the portion of the individual's bony anatomy and skin proximate the bony anatomy can be registered with the computer aided surgical navigation system, the computer functionality adapted to generate information corresponding to the position and orientation of the individual's bony anatomy and skin proximate the bony anatomy by receiving information from the sensor sensing the position and orientation of the first surgical reference, the computer functionality adapted to calculate a suggested incision path and length on the skin proximate the bony anatomy based upon the information generated by the computer functionality corresponding to the position and orientation of the individual's bony anatomy; at least one second surgical reference adapted to be associated with an incision device, wherein the incision device is a cutting device or a marking device, the at least one second surgical reference adapted to be registered with the computer aided surgical navigation system, the computer functionality adapted to generate information corresponding to the position of the incision device by receiving information from the sensor sensing the position and orientation of the second surgical reference; and wherein the computer aided surgical navigation system is adapted to compare the suggested incision path and length with the information corresponding to the position of the incision device.

A system for guiding an incision on a skin surface further characterized in that the system includes a display, the display adapted to compare the suggested incision path and length with the information concerning the position of the incision device by displaying the suggested incision path and length and the position of the incision device.

A system for guiding an incision on a skin surface further characterized in that the display comprises a semi-transparent display or a monitor.

A system for guiding an incision on a skin surface further characterized in that the display comprises a semi-transparent display, the semi-transparent display associated with at least one third surgical reference, the at least one third surgical reference adapted to be registered with the computer aided surgical navigation system, the computer functionality adapted to generate information corresponding to the position and orientation of the semi-transparent display by receiving information from the sensor sensing the position and orientation of the third surgical reference.

A system for guiding an incision on a skin surface of further characterized in that the computer aided surgical navigation system further comprises feedback functionality adapted to provide feedback if the incision device deviates from the suggested incision path.

A system for guiding an incision on a skin surface further characterized in that the feedback functionality is adapted to provide visual feedback if the incision device deviates from the suggested incision path.

A system for guiding an incision on a skin surface further characterized in that the feedback functionality comprises a display adapted to display the suggested incision and a path traveled by the incision device.

A system for guiding an incision on a skin surface further characterized in that the system also includes a projector, the projector: adapted to project an image of the suggested incision path onto the individual; and adapted to provide visual feedback if the incision device deviates from the suggested incision path by altering the image if the incision device deviates from the suggested incision path.

A system for guiding an incision on a skin surface further characterized in that the feedback functionality is adapted to provide audible feedback if the incision device deviates from the suggested incision path.

A system for guiding an incision on a skin surface, characterized in that the system comprises: a computer aided surgical navigation system, comprising: a sensor adapted to sense the position and orientation of at least one surgical reference associated with a structure to be referenced; and computer functionality adapted to receive information from the sensor about position and orientation of the surgical reference and generate information corresponding to the position and orientation of the structure to be referenced to which the surgical reference is associated; at least one first surgical reference adapted to be associated with a portion of an individual's bony anatomy and skin proximate the bony anatomy such that the position and orientation of the portion of the individual's bony anatomy and skin proximate the bony anatomy can be registered with the computer functionality, the computer functionality adapted to generate information corresponding to the position and orientation of the individual's bony anatomy and skin proximate the bony anatomy by receiving information from the sensor sensing the position and orientation of the first surgical reference, the computer functionality adapted to calculate a suggested incision path and length based upon the information generated by the computer functionality corresponding to the position and orientation of the individual's bony anatomy and skin proximate the bony anatomy; at least one second surgical reference adapted to be associated with an incision device, wherein the incision device is a cutting device or a marking device, the at least one second surgical reference and the incision device adapted to be registered with the computer aided surgical navigation system, the computer functionality adapted to generate information corresponding to the position of the incision device by receiving information from the sensor sensing the position and orientation of the second surgical reference; wherein the computer aided surgical navigation system is adapted to compare the suggested incision with the information corresponding to the position of the incision device; and wherein the computer aided surgical navigation system further comprises feedback functionality adapted to provide feedback if the incision device deviates from the suggested incision.

A system for guiding an incision on a skin surface further characterized in that the system also includes a projector, the projector adapted to project an image of the suggested incision path onto the individual and adapted to provide visual feedback by altering the image if the incision device deviates from the suggested incision path.

A system for guiding an incision on a skin surface further characterized in that the system also includes a display, the display adapted to display an image of the suggested incision path and display at least a portion of a path followed by the incision device.

A system for guiding an incision on a skin surface, characterized in that the system comprises: a computer aided surgical navigation system, comprising: a sensor adapted to sense the position and orientation of at least one surgical reference associated with a structure to be referenced; and computer functionality adapted to receive information from the sensor about position and orientation of the surgical reference and generate information corresponding to the position and orientation of the structure to be referenced to which the surgical reference is associated; at least one first surgical reference adapted to be associated with a portion of an individual's bony anatomy and skin proximate the bony anatomy such that the position and orientation of the portion of the individual's bony anatomy and skin proximate the bony anatomy can be registered with the computer functionality, the computer functionality adapted to generate information corresponding to the position and orientation of the individual's bony anatomy and skin proximate the bony anatomy by receiving information from the sensor sensing the position and orientation of the first surgical reference, the computer functionality adapted to calculate a suggested incision path and length based upon the information generated by the computer functionality corresponding to the position and orientation of the individual's bony anatomy and skin proximate the bony anatomy; at least one second surgical reference adapted to be associated with an indicator device, the at least one second surgical reference and indicator device adapted to be registered with the computer aided surgical navigation system, the computer functionality adapted to generate information corresponding to the position of the indicator device by receiving information from the sensor sensing the position and orientation of the second surgical reference; and wherein the computer aided surgical navigation system assists the indicator device in outputting the suggested incision path and length onto the skin surface based upon the information corresponding to the position of the indicator device and the information corresponding to the position and orientation of the individual's bony anatomy and skin proximate the bony anatomy.

A system for guiding an incision on a skin surface further characterized in that the indicator device comprises a marking device.

A system for guiding an incision on a skin surface further characterized in that the indicator device comprises a projector adapted to project an image of the suggested incision path onto the individual.

### BRIEF DESCRIPTION OF THE DRAWING

FIG. 1 shows a schematic view of a system for performing a minimally invasive incision according to a first embodiment of the present invention.

### DETAILED DESCRIPTION

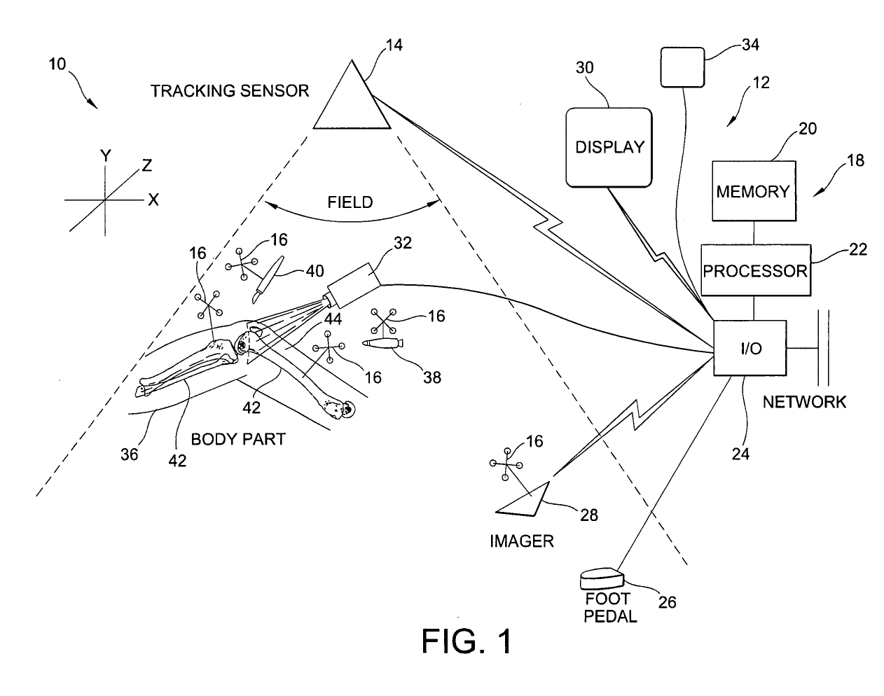

FIG. 1 shows a schematic view of a system 10 for performing a minimally invasive incision according to a first embodiment of the present invention. System 10 may provide guidance for performing a surgical incision by tracking and comparing the real time position of an incision device with a calculated suggested incision. The suggested incision path and length may be calculated using information obtained by tracking the position and orientation of the individual's anatomy 36 to be incised (including the relevant bony anatomy 42 and skin proximate the bony anatomy 44). As shown in FIG. 1, system 10 may include, sensor 14, computer functionality 18 (which may include memory functionality 20, processing functionality 22 and input/output functionality 24), display 30, projector 32, other output device 34, foot pedal 26, imaging device 28, surgical references 16, marking device 38 and/or cutting device 40. System 10 does not require all of these items, systems 10 according to various embodiments of the present invention may have other combinations of these or other items. For example, in a preferred embodiment, it is only necessary to associate either the marking device 38 or the cutting device 40 with a surgical reference 16, not both. In some embodiments, marking device 38 is not necessary and the cutting device 40 is associated with a surgical reference 16.

FIG. 1 shows system 10 used to perform a minimally invasive incision on a knee. However, system 10 is not limited to incisions proximate the knee, system 10 can be used to guide minimally invasive incisions on any desired portion of an individual's anatomy.

In the embodiment shown in FIG. 1, system 10 includes a computer aided surgical navigation system 12, such as the TREON™, ION™ or VECTORVISION™ systems described above. Computer aided surgical navigation system 12 may include a sensor 14 and computer functionality 18. Sensor 14 may be any suitable sensor, such as the ones described above or other sensors, capable of detecting the position and/or orientation of surgical references 16. In a preferred embodiment, sensor 14 emits infrared light and detects reflected infrared light to sense the position and/or orientation of surgical references 16.

Surgical reference 16 may be any device that can be secured to a structure to be referenced and detected by a sensor 14 such that the position and/or orientation of the surgical reference 16 can be detected. Suitable surgical references 16 may include, but are not limited to, fiducials secured to the bony anatomy by a pin or screw; modular fiducials secured to a platform or other structure; magnetic fiducials; quick release fiducials; adjustable fiducials; electromagnetic emitters; radio frequency emitters; LED emitters or any other surgical reference suitable for tracking by a computer assisted surgical navigation system. These and other suitable surgical references 16 are described in the documents incorporated by reference into this document.

In the embodiment shown in FIG. 1, sensor 14 may communicate information to the computer functionality 18 corresponding to the position and orientation of a surgical reference 16. Computer functionality 18, using memory functionality 20 and/or processing functionality 22 may then calculate the position and/or orientation of the structure to be referenced associated with the surgical reference 16 based on the sensed position and orientation of the surgical reference 16.

In the embodiment shown in FIG. 1, surgical references 16 are associated with structures to be referenced including an individual's body part 36 (including bony anatomy 42 and skin proximate the bony anatomy 44), marking device 38 and cutting device 40. For example, surgical reference 16 may be associated with the bony anatomy 42 and proximate skin 44 by first securely fastening surgical reference 16 to the bony anatomy 42. This may be done in any suitable and/or desirable manner, including securing the surgical reference 16 to the bony anatomy 42 in ways described above. Subsequently, imaging, such as fluoroscopy, X-ray, or other information corresponding to the bony anatomy 42, proximate skin 44 and other structure may be obtained and associated with the position and/or orientation of the surgical reference 16 secured to the bony anatomy 42. As shown in FIG. 1, such information may be obtained and associated using an imaging device 28, such as a fluoroscope associated with another surgical reference 16, or may be obtained by any other desirable and/or suitable method. Associating surgical reference 16 with the bony anatomy 42 and proximate skin 44 in this manner may allow system 10 to track and display the position and orientation of bony anatomy 42 and proximate skin 44 based on the sensed position and orientation of surgical reference 16.

Surgical references 16 may also be associated with other items, such as the marking device 38 and cutting device 40 shown in FIG. 1, which the computer functionality 18 already has information on, such as wire-frame data. In such circumstances, a probe or other suitable device may be used to register the position and orientation of the surgical reference into the computer aided surgical navigation system allowing the position and/or orientation of the marking device 38 or cutting device 40 to be associated with the sensed position and orientation of the surgical reference 16. In some embodiments of the present invention, it is only necessary to track the position of the incision device. In some preferred embodiments, the tip of the incision device is what is tracked and compared with the suggested incision. In other embodiments, it may be preferable to track the position and orientation of the incision device. For example, it may be desirable to have the cutting device 40 enter the skin 44 at a certain angle. In such embodiments, it may be desirable to track the position and orientation of the cutting device 40 such that the entry angle of the cutting device 40 can be determined.

Using the information generated corresponding to the position and orientation of the bony anatomy 42 and proximate skin 44, the computer functionality 18 shown in FIG. 1 may be used to calculate a suggested incision path and length on the skin 44 proximate the bony anatomy 42 for a desired surgical procedure. In some embodiments, the computer functionality 18 may do this automatically, using appropriate software. For instance, the software may be programmed to identify bone and tissue structures in the obtained imaging relevant to the incision or incisions necessary for a desired procedure and calculate a suggested incision path and length based on the identified structures. In other embodiments, the surgeon may use a display 30 or other suitable device to manually designate the location of the relevant bone and tissue structures such that the computer functionality 18 can calculate the suggested incision path. In still other embodiments, the surgeon or other user may designate the suggested incision path manually, such as by using a light pen, mouse or other suitable device in conjunction with a display 30 showing the bony anatomy 42 and proximate skin 44.

In some embodiments, computer functionality 18 may be adapted to update the suggested incision path and length in real time if the position or orientation of the body part 36 changes. By tracking the position and orientation of the surgical reference or references 16 associated with the bony anatomy 42 and proximate skin 44 the computer functionality 18 may adjust the suggested incision path and/or length to compensate for changes in position and/or orientation of the body part 36.

In other embodiments, such as in embodiments where the surgeon has manually designated the suggested incision, system 10 may provide a visible or audible warning if the body part 36 changes in position and/or orientation, potentially rendering the suggested incision inaccurate. In other embodiments, the computer functionality 18 and/or software may be adapted to update the surgeon or other user's manual designation automatically if the position and/or orientation of the body part 36 changes.

Regardless of how the suggested incision path and length is determined, the computer functionality can output the suggested incision in any desirable and/or suitable manner. In one embodiment, a display 30 displays a visual representation of the suggested incision shown overlaying image data of the bony anatomy 42 and proximate skin 44, the position and orientation of which is determined by the computer assisted surgical navigation system 12. Display 30 may be any suitable device including a monitor or a semi-transparent display. In some embodiments, the semi-transparent display may be a “heads-up display” worn by the surgeon or other user. In other embodiments, a projector 32 or other suitable device projects the suggested incision onto the individual's skin 44 using a projector 32 or other suitable device. In some embodiments where a projector 32 or a semi-transparent display is used, the projector 32 or semi-transparent display may be associated with a surgical reference 16 to allow the suggested incision path and length to be correlated with the position and orientation of the semi-transparent display or projector 32. In these embodiments, even if the position of the projector 32 or semi-transparent display is altered, either inadvertently or intentionally, the suggested incision as projected onto the proximate skin 44 may remain accurate because the computer functionality 18 can modify the image projected by the projector 32 to account for changes of position or orientation of the projector 32. In embodiments where projector 32, other suitable projection device, or semi-transparent display is associated with a surgical reference 16, it may be preferable to not associate marking device 38 and/or cutting device 40 with a surgical reference 16. Rather, the projector 32 or other device associated with surgical reference 16 may provide enough guidance to perform the incision or other procedure.

In the embodiment shown in FIG. 1, the computer aided surgical navigation system 12 is not only used to determine the suggested incision path and length, but to also track the position of an incision device, such as a marking device 38 or a cutting device 40. By tracking and comparing the incision device and the suggested incision path in real time, system 10 may assist a surgeon or other user in performing a minimally invasive surgical incision.

Marking device 38 or cutting device 40 may be associated with surgical references 16 such that the computer aided surgical navigation system 12 can track the position and/or orientation of the incision device. Marking device 38 may be a sterile, surgical marker or other appropriate device that may be used by a surgeon or other individual to mark the suggested incision path and length on the individual's anatomy. Cutting device 40 may be a scalpel or any other desired instrument for performing the incision. In embodiments where a marking device 38 is used, cutting device 40 does not also have to be tracked by the surgical navigation system 12, however, it may be tracked if the surgeon or other user desires.

In some embodiments, a projector 32 may project the suggested incision path and length onto the anatomy of the individual. Preferably, projector 32 uses a scanning laser to project the suggested incision path and length onto the individual's anatomy. However, projector 32 may also be other suitable devices. Projector 32 may be used in conjunction with or without cutting device 40 and/or marking device 38 in the performance of the incision. The surgeon or other user may follow the suggested incision path and length projected onto the skin 44 to either make the incision with the incision device 40 or to mark where the incision will take place with the marker 38. However, whether or not the use of projector 32 is desired may depend on what other instruments, including cutting device 40 and/or marking device 38, are associated with surgical references 16.

In some embodiments of the present invention, computer aided surgical navigation system 12 tracks the position and/or orientation of marking device 38 and/or cutting device 40 in real time. Consequently, the computer functionality 18 may compare the sensed position and/or orientation of the incision device with the calculated suggested incision path and length during the marking and/or cutting process. In some embodiments, the computer aided surgical navigation system 12, using the input/output functionality 24, may output the comparison to aid the surgeon in performing the incision. However, not all embodiments of the present invention require the output of the comparison. In some embodiments, the computer functionality 18, using the input/output functionality 24, may simply output a warning if, while comparing the position and/or orientation of the incision device with the suggested incision, the incision device deviates from the suggested incision.

In some embodiments, the comparison may be outputted on a display 30 or other appropriate output device 34. The display 30 may display an overlay of the information corresponding to the real time position of the incision device and/or information corresponding to the suggested incision with the image data of the bony anatomy 42 and/or proximate skin 44, shown correctly oriented with respect to their actual positions and/or orientations. In some embodiments, display 30 may be a monitor. In other embodiments, the display 30 may be a semi-transparent display, such as a heads-up display worn by the surgeon or other user, or any other desired display. In some embodiments where the display is a semi-transparent display, the semi-transparent display may be associated with a surgical reference 16 or other fiducial functionality to allow the suggested incision path and/or actual position of the incision device to be displayed correctly relative to the portion of anatomy viewed through the semi-transparent display in real time.

System 10 may also provide feedback to the surgeon or other user if the incision device, during either marking or cutting, deviates from the suggested incision path. System 10 may be calibrated with any desired tolerance for an acceptable deviation. System 10 may provide feedback in any desirable and/or suitable manner including visual feedback, audio feedback, tactile feedback or any other type of feedback suitable for informing the surgeon of when the cut or mark deviates from the suggested incision.

In some embodiments, the real time comparison of the suggested incision with the actual position of the incision device on the display 30 provides visual feedback. In some of these embodiments, a visible warning may be displayed if the incision device deviates from the suggested incision. The visible warning may take any desired form, such as a flashing light, change in color or other aspect of the displayed suggested incision or displayed incision device or any other appropriate warning. In embodiments where a projector 32 is used, the projected image may change in aspect to provide a visual waning of incision device deviation.

Changes, modifications, additions, deletions and other alternations may be made to the embodiments described above without departing from the spirit or scope of the invention as defined in the following claims.

1. A method of performing an incision on a skin surface using a computer aided surgical navigation system, the system comprising: a sensor adapted to sense the position and orientation of at least one surgical reference associated with a structure to be referenced; and computer functionality adapted to receive information from the sensor about position and orientation of the surgical reference and generate information corresponding to the position and orientation of the structure to be referenced to which the surgical reference is associated, the method of guiding the surgical incision comprising:
(a) associating at least one first surgical reference with a portion of an individual's bony anatomy and skin proximate the bony anatomy;
(b) registering the position and orientation of the portion of the individual's bony anatomy and skin proximate the bony anatomy with the computer aided surgical navigation system such that the computer functionality can generate information corresponding to the position and orientation of the individual's bony anatomy and skin proximate the bony anatomy by receiving information from the sensor sensing the position and orientation of the first surgical reference;
(c) calculating a suggested incision path and length based upon the information generated by the computer functionality corresponding to the position and orientation of the individual's bony anatomy and skin proximate the bony anatomy;
(d) associating at least one second surgical reference with an incision device, wherein the incision device is a cutting device or a marking device;
(e) registering the position of the incision device with the computer aided surgical navigation system such that the computer functionality can generate information corresponding to the position of the incision device by receiving information from the sensor sensing the position and orientation of the second surgical reference; and
(f) using the incision device in the performance of the incision, wherein the computer aided surgical navigation system provides guidance based on comparing the suggested incision path and length with the information corresponding to the position and orientation of the incision device as generated by the computer functionality by receiving information from the sensor sensing the position and orientation of the second surgical reference.

2. The method of performing an incision on a skin surface using a computer aided surgical navigation system of claim 1, wherein the computer aided surgical navigation system providing guidance further comprises a display associated with the computer aided surgical navigation system displaying the suggested incision path and length and the position of the incision device.

3. The method of performing an incision on a skin surface using a computer aided surgical navigation system of claim 2, wherein displaying the suggested incision path and length and the position of the incision device further comprises a semi-transparent display displaying the suggested incision path and length and the position of the of the incision device or a monitor displaying the suggested incision path and length and the position of the of the incision device.

4. The method of performing an incision on a skin surface using a computer aided surgical navigation system of claim 1, further comprising providing feedback if the incision device deviates from the suggested incision.

5. The method of performing an incision on a skin surface using a computer aided surgical navigation system of claim 4, wherein providing feedback comprises providing a visible warning if the incision deviates from the suggested incision.

6. The method of performing an incision on a skin surface using a computer aided surgical navigation system of claim 5, wherein providing a visible warning if the incision deviates from the suggested incision comprises displaying a comparison of the suggested incision path and length with at least a portion of a path traveled by the incision device.

7. The method of performing an incision on a skin surface using a computer aided surgical navigation system of claim 5, further comprising projecting an image of the suggested incision onto the individual; and wherein providing a visible warning if the incision deviates from the suggested incision comprises altering the image if the incision deviates from the suggested incision.

8. The method of performing an incision on a skin surface using a computer aided surgical navigation system of claim 4, wherein providing feedback further comprises providing an audible warning if the incision deviates from the suggested incision path.

9. The method of performing an incision on a skin surface using a computer aided surgical navigation system of claim 1, further comprising: using the incision to access the interior of the individual to install an orthopaedic implant; and installing the orthopaedic implant.

10. A system for guiding an incision on a skin surface, comprising:
(a) a computer aided surgical navigation system, comprising:
(i) a sensor adapted to sense the position and orientation of at least one surgical reference associated with a structure to be referenced; and
(ii) computer functionality adapted to receive information from the sensor about position and orientation of the surgical reference and generate information corresponding to the position and orientation of the structure to be referenced to which the surgical reference is associated;
(b) at least one first surgical reference adapted to be associated with a portion of an individual's bony anatomy and skin proximate the bony anatomy such that the position and orientation of the portion of the individual's bony anatomy and skin proximate the bony anatomy can be registered with the computer aided surgical navigation system, the computer functionality adapted to generate information corresponding to the position and orientation of the individual's bony anatomy and skin proximate the bony anatomy by receiving information from the sensor sensing the position and orientation of the first surgical reference, the computer functionality adapted to calculate a suggested incision path and length on the skin proximate the bony anatomy based upon the information generated by the computer functionality corresponding to the position and orientation of the individual's bony anatomy;
(c) at least one second surgical reference adapted to be associated with an incision device, wherein the incision device is a cutting device or a marking device, the at least one second surgical reference adapted to be registered with the computer aided surgical navigation system, the computer functionality adapted to generate information corresponding to the position of the incision device by receiving information from the sensor sensing the position and orientation of the second surgical reference; and
(d) wherein the computer aided surgical navigation system is adapted to compare the suggested incision path and length with the information corresponding to the position of the incision device.

11. The system for guiding an incision on a skin surface of claim 10, further comprising a display, the display adapted to compare the suggested incision path and length with the information concerning the position of the incision device by displaying the suggested incision path and length and the position of the incision device.

12. The system for guiding an incision on a skin surface of claim 11, wherein the display comprises a semi-transparent display or a monitor.

13. The system for guiding an incision on a skin surface of claim 12, wherein the display comprises a semi-transparent display, the semi-transparent display associated with at least one third surgical reference, the at least one third surgical reference adapted to be registered with the computer aided surgical navigation system, the computer functionality adapted to generate information corresponding to the position and orientation of the semi-transparent display by receiving information from the sensor sensing the position and orientation of the third surgical reference.

14. The system for guiding an incision on a skin surface of claim 10, wherein the computer aided surgical navigation system further comprises feedback functionality adapted to provide feedback if the incision device deviates from the suggested incision path.

15. The system for guiding an incision on a skin surface of claim 14, wherein the feedback functionality is adapted to provide visual feedback if the incision device deviates from the suggested incision path.

16. The system for guiding an incision on a skin surface of claim 15, wherein the feedback functionality comprises a display adapted to display the suggested incision and a path traveled by the incision device.

17. The system for guiding an incision on a skin surface of claim 15, further comprising a projector, the projector: adapted to project an image of the suggested incision path onto the individual; and adapted to provide visual feedback if the incision device deviates from the suggested incision path by altering the image if the incision device deviates from the suggested incision path.

18. The system for guiding an incision on a skin surface of claim 15, wherein the feedback functionality is adapted to provide audible feedback if the incision device deviates from the suggested incision path.

19. A system for guiding an incision on a skin surface, comprising:
(a) a computer aided surgical navigation system, comprising:
(i) a sensor adapted to sense the position and orientation of at least one surgical reference associated with a structure to be referenced; and
(ii) computer functionality adapted to receive information from the sensor about position and orientation of the surgical reference and generate information corresponding to the position and orientation of the structure to be referenced to which the surgical reference is associated;
(b) at least one first surgical reference adapted to be associated with a portion of an individual's bony anatomy and skin proximate the bony anatomy such that the position and orientation of the portion of the individual's bony anatomy and skin proximate the bony anatomy can be registered with the computer functionality, the computer functionality adapted to generate information corresponding to the position and orientation of the individual's bony anatomy and skin proximate the bony anatomy by receiving information from the sensor sensing the position and orientation of the first surgical reference, the computer functionality adapted to calculate a suggested incision path and length based upon the information generated by the computer functionality corresponding to the position and orientation of the individual's bony anatomy and skin proximate the bony anatomy;
(c) at least one second surgical reference adapted to be associated with an incision device, wherein the incision device is a cutting device or a marking device, the at least one second surgical reference and the incision device adapted to be registered with the computer aided surgical navigation system, the computer functionality adapted to generate information corresponding to the position of the incision device by receiving information from the sensor sensing the position and orientation of the second surgical reference;
(d) wherein the computer aided surgical navigation system is adapted to compare the suggested incision with the information corresponding to the position of the incision device; and
(e) wherein the computer aided surgical navigation system further comprises feedback functionality adapted to provide feedback if the incision device deviates from the suggested incision.

20. The system for guiding an incision on a skin surface of claim 19, further comprising a projector, the projector adapted to project an image of the suggested incision path onto the individual and adapted to provide visual feedback by altering the image if the incision device deviates from the suggested incision path.

21. The system for guiding an incision on a skin surface of claim 19, further comprising a display, the display adapted to display an image of the suggested incision path and display at least a portion of a path followed by the incision device.

22. A system for guiding an incision on a skin surface, comprising:
(a) a computer aided surgical navigation system, comprising:
(i) a sensor adapted to sense the position and orientation of at least one surgical reference associated with a structure to be referenced; and
(ii) computer functionality adapted to receive information from the sensor about position and orientation of the surgical reference and generate information corresponding to the position and orientation of the structure to be referenced to which the surgical reference is associated;
(b) at least one first surgical reference adapted to be associated with a portion of an individual's bony anatomy and skin proximate the bony anatomy such that the position and orientation of the portion of the individual's bony anatomy and skin proximate the bony anatomy can be registered with the computer functionality, the computer functionality adapted to generate information corresponding to the position and orientation of the individual's bony anatomy and skin proximate the bony anatomy by receiving information from the sensor sensing the position and orientation of the first surgical reference, the computer functionality adapted to calculate a suggested incision path and length based upon the information generated by the computer functionality corresponding to the position and orientation of the individual's bony anatomy and skin proximate the bony anatomy;
(c) at least one second surgical reference adapted to be associated with an indicator device, the at least one second surgical reference and indicator device adapted to be registered with the computer aided surgical navigation system, the computer functionality adapted to generate information corresponding to the position of the indicator device by receiving information from the sensor sensing the position and orientation of the second surgical reference; and
(d) wherein the computer aided surgical navigation system assists the indicator device in outputting the suggested incision path and length onto the skin surface based upon the information corresponding to the position of the indicator device and the information corresponding to the position and orientation of the individual's bony anatomy and skin proximate the bony anatomy.

23. The system for guiding an incision on a skin surface of claim 22, wherein the indicator device comprises a marking device.

24. The system for guiding an incision on a skin surface of claim 22, wherein the indicator device comprises a projector adapted to project an image of the suggested incision path onto the individual.
